(19) United States
(12) Patent Application Publication
(10) Pub. No.: US 2008/0312245 A1
(43) Pub. Date: Dec. 18, 2008

(54) Methods and compositions for treating acne and other infectious diseases

(76) Inventor: Longyan Xiao, Ji'an (CN)

Correspondence Address: Longyan Xiao, 922 Moreno Ave., Palo Alto, CA, 94303, US

(21) Appl. No.: 11/973,644

(22) Filed: Oct. 9, 2007

(30) Foreign Application Priority Data
Jun. 12, 2007 (CN) CN200710126131.1

(51) Int. Cl. A61K 31/496; A61P 31/00

(52) U.S. Cl. 514/252.2

(57) ABSTRACT

The present invention provides a composition comprising metronidazole, ketoconazole, and lincomycin and methods of treating infectious skin diseases such as acne by topically administering the composition.

### RELATED APPLICATIONS

This application claims priority benefit to Chinese Patent Application Serial No. 200710126131.4, filed on Jun. 12, 2007.

### TECHNICAL FIELD

This application relates to methods and compositions that are useful for treating acne and other infectious skin diseases.

### BACKGROUND

Acne is a common inflammatory disease which is very common at puberty and which occurs in skin areas where sebaceous glands are largest, most numerous, and most active. In its mild form, acne is a superficial disorder which is evidence by slight, spotty irritations. Occasionally, pilosebaceous follicles occurs and results in the formation of pustules, infected cysts, and in extreme cases, canalizing inflamed and infected sacs, which may become extensive and leave permanent, disfiguring scars. There is a need for treatment of both mild and severe cases of acne.

The disclosures of all publications, patents, patent applications and published patent applications referred to herein are hereby incorporated herein by reference in their entirety.

### BRIEF SUMMARY OF THE INVENTION

The present invention in one aspect provides a composition for treatment of an infectious skin disease (such as acne) in an individual by topical administration, comprising metronidazole, ketoconazole, and lincomycin.

In some embodiments, the metronidazole in the composition is about 1% to about 30%, including for example about 5% to about 25%, about 20%, or about 10% (w/w). In some embodiments, the metronidazole in the composition is at least about 1%, including for example at least about any of 2%, 5%, 10%, 15%, 20%, or 25%.

In some embodiments when the composition is a liquid compositions (such as a liquid suspension), the metronidazole in the composition is about 0.1% to about 2% (w/v), including for example about 0.2% to about 1%, or 0.5% (w/v). In some embodiments, the metronidazole in the composition is at least about any of 0.1%, 0.2%. 0.3%, 0.4%, or 0.5% (w/v).

In some embodiments, the ketoconazole in the composition is about 0.5% to about 10% (w/w), including for example about 1% to about 9%, about 5% to about 8%, or about 8%. In some embodiments, the ketoconazole in the composition is at least about 0.5%, including for example at least about any of 1%, 2%, 5%, 8%, or 10%.

In some embodiments when the composition is a liquid composition (such as a liquid suspension), the ketoconazole in the composition is about 0.05% to about 0.5%, including for example about any of 0.1% to 1%, 0.1% to 0.5%, or 0.2% (w/v). In some embodiments, the ketoconazole in the composition is at least about any of 0.05%, 0.1%, or 0.2%.

In some embodiments, the lincomycin in the composition is about 10% to about 80%, including for example about 20% to about 80%, about 30% to about 80%, about 50% to about 75%, about 72%. In some embodiments, the lincomycin in the composition is at least about any of 5%, 10%, 20%, 30%, 40%, 50%, 60%, 70%, or 80%.

In some embodiments when the composition is a liquid composition (such as a liquid suspension), the lincomycin is present in the. composition for about 0.5% to about 5% (w/v), including for example about 0.8% to about 3%, about 1% to about 2%, or 1.8% (w/v). In some embodiments, the lincomycin is present in the composition for at least about any of 0.5%, 0.8%, 1%, 1.5%, or 1.8%.

In some embodiments, the metronidazole is present in the composition for about 5% to about 20% (w/w) (such as about 10% to about 20%), the ketoconazole is present in the composition for about 1% to about 10% (w/w) (such as about 5% to about 8%), and lincomycin is present in the composition for about 20% to about 72% (w/v) (such as about 50% to about 72%, or about 70% to about 72%).

In some embodiments when the composition is a liquid composition (such as a liquid suspension), the metronidazole is present in the composition for about 0.05% to about 0.5% (w/v) (such as about 0.5%), ketoconazole is present in the composition for about 0.05% to about 0.5% (w/v)(such as about 0.2%), and lincomycin is present in the composition for about 0.5% to about 5% (w/v) (such as about 1.8%).

In some embodiments, the metronidazole, ketoconazole, and lincomycin are present in the composition for about 0.5%, 0.2%, and 1.8% (w/v), respectively.

In some embodiments, the metronidazole, ketoconazole, and lincomycin are present in a synergistic ratio. For example, in some embodiments, the weight ratio of metronidazole and ketoconazole in the composition is about 1:5 to about 5:1, including for example about any of 1:2, 1:1, 2:1, 3:2, 5:2, or 5:1. In some embodiments, the weight ratio of lincomycin and ketoconazole in the composition is about 1:10 to a bout 10:1, including for example about any of 1:5, 1:1, 2:1, 3:1, 4:1, 5:1, 6:1, 7:1, 8:1, 9:1, or 10:1. In some embodiments, the weight ratio of metronidazole, ketoconazole, and lincomycin in the composition is about 2.5:1:9. In some embodiments, the ratio of metronidazole, ketoconazole, and lincomycin in the composition is about 2:5:1:4.5.

The composition comprising metronidazole, ketoconazole, and lincomycin can be in any suitable forms. For example, in some embodiments, the composition is in the form of a suspension. In some embodiments, the composition is in the form of an aqueous gel, such as an aqueous alcohol gel. In some embodiments, the composition is in the form of a cream. In some embodiments, the composition is in the form of an ointment. In some embodiments, the composition is in the form of an emulsion. In some embodiments, the composition is in the form of a liposomal composition.

In some embodiments, the composition further comprises a dermatologically acceptable carrier.

In another aspect, there is provided a method of treating an infectious skin disease (such as acne) in an individual, comprising topically administering to the affected area of the individual an effective amount of any of the compositions described herein. In some embodiments, the method further comprises applying ionized vapor to the affected area prior to the administration of the composition. In some embodiments, the method further comprises the step of removing the nodule prior to administration of the composition. In some embodiments, the composition is administered with the aid of ultrasound. In some embodiments, the individual is less than about 30 years old.

### DETAILED DESCRIPTION OF THE INVENTION

The present invention provides compositions and methods that are useful for treating acne and other infectious skin diseases. Specifically, inventor has invented a new composition comprising metronidazole, ketoconazole, and lincomycin and found that the composition, when applied topically to affected skin areas of an individual with acne, work surprising well in treating acne. Furthermore, inventor found that use of ionized evaporation and/or ultrasound delivery method facilitated absorption of the composition and increases efficacy of treating acne. The compositions and methods disclosed herein have been used in China to treat inventor's patients and have been shown to be highly effective.

Accordingly, the present invention in one aspect provides a composition comprising metronidazole, ketoconazole, and lincomycin.

In another aspect, the present invention provides a method of treating or preventing an infectious skin disease (such as acne) in an individual by topically administering to the individual an effective amount of a composition comprising metronidazole, ketoconazole, and lincomycin.

As used herein, the singular form “a”, “an”, and “the” includes plural references unless indicated otherwise.

It is understood that aspect and embodiments of the invention described herein include “consisting” and/or “consisting essentially of” aspects and embodiments.

### Compositions Comprising Metronidazole, Ketoconazole and Lincomycin

The present invention provides a composition comprising metronidazole, ketoconazole, and lincomycin.

Metronidazole is an antiprotozoal and anti-bacterial agent: Chemically, metronidazole is 2-methyl-5-nitro-1H-imidazole-1-ethanol. Metronidazole is slightly soluble in alcohol and has a solubility in water of 10 mg/ml. at 20° C. Metronidazole is a member of the imidazole class of antibacterial. Metronidazole used herein includes salts of metronidazole.

Ketoconazole as mentioned hereinabove is the generic name of the compound (.±.)-cis-1-acetyl-4-[4-[[2-(2,4-dichlorophenyl)-2-(1H-imidazol-1-ylmethyl)-1,3-dioxolan-4-yl]methoxy]phenyl]piperazine. The compound ketoconazole used in the method of the present invention is a known antifungal agent and its preparation as well as its pharmacological properties are described in U.S. Pat. No. 4,335,125.

The compound ketoconazole used herein includes pharmaceutically acceptable acid addition salt forms, the latter being conveniently obtained by treating the base form with an appropriate acid. Appropriate acids comprise, for example, inorganic acids such as hydrohalic acids, e.g. hydrochloric or hydrobromic acid; sulfuric acid; nitric acid; phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. The term acid addition salt form as used hereinabove also comprises the solvates which the compound ketoconazole and its acid addition salts are able to form. Examples of such solvates are e.g. the hydrates, alcoholates and the like.

Lincomycin is a derivative of the amino acid trans-L-4-.alpha.-propyl-hygrinic acid coupled to a derivative of an octose substituted by a methylmercaptyl group. Clndamycin is the 7-deoxy, 7-chloro derivative of lincomycin, and is otherwise known as methyl 7-chloro-6,7,8,trideoxy-6-[[(1-methyl-4-propyl-2-pyrrolidinyl)carbonyl]amino]-1-thio-L-threo-.alpha.-D-galacto-octopyranoside. The lincomycin antibiotics are described in U.S. Pat. Nos. 3,475,407; 3,509,127; 3,544,551 and 3,513,155.

In some embodiments, the metronidazole in the composition is about 1% to about 30%, including for example about 5% to about 25%, about 20%, or about 10% (w/w). In some embodiments, the metronidazole in the composition is at least about 1%, including for example at least about any of 2%, 5%, 10%, 15%, 20%, or 25%.

In some embodiments when the composition is a liquid compositions (such as a liquid suspension), the metronidazole in the composition is about 0.1% to about 2% (w/v), including for example about 0.2% to about 1%, or 0.5% (w/v). In some embodiments, the metronidazole in the composition is at least about any of 0.1%, 0.2%. 0.3%, 0.4%. or 0.5% (w/v).

In some embodiments, the ketoconazole in the composition is about 0.5% to about 10% (w/w), including for example about 1% to about 9%, about 5% to about 8%, or about 8%. In some embodiments, the ketoconazole in the composition is at least about 0.5%, including for example at least about any of 1%, 2%, 5%, 8%, or 10%.

In some embodiments when the composition is a liquid composition (such as a liquid suspension), the ketoconazole in the composition is about 0.05% to about 0.5%, including for example about any of 0.1% to 1%, 0.1% to 0.5%, or 0.2% (w/v). In some embodiments, the ketoconazole in the composition is at least about any of 0.05%, 0.1%, or 0.2%.

In some embodiments, the lincomycin in the composition is about 10% to about 80%, including for example about 20% to about 80%, about 30% to about 80%, about 50% to about 75%, about 72%. In some embodiments, the licomycin in the composition is at least about any of 5%, 10%, 20%, 30%, 40%, 50%, 60%, 70%, or 80%.

In some embodiments when the composition is a liquid composition (such as a liquid suspension), the lincomycin is present in the composition for about 0.5% to about 5% (w/v), including for example about 0.8% to about 3%, about 1% to about 2%, or 1.8% (w/v). In some embodiments, the lincomycin is present in the composition for at least about any of 0.5%, 0.8%, 1%, 1.5%, or 1.8%.

In some embodiments, the metronidazole is present in the composition for about 5% to about 20% (w/w) (such as about 10% to about 20%), the ketoconazole is present in the composition for about 1% to about 10% (w/w) (such as about 5% to about 8%), and lincomycin is present in the composition for about 20% to about 72% (w/v) (such as about 50% to about 72%, or about 70% to about 72%).

In some embodiments when the composition is a liquid composition (such as a liquid suspension), the metronidazole is present in the composition for about 0.05% to about 0.5% (w/v) (such as about 0.5%), ketoconazole is present in the composition for about 0.05% to about 0.5% (w/v)(such as about 0.2%), and lincomycin is present in the composition for about 0.5% to about 5% (w/v) (such as about 1.8%).

In some embodiments, the metronidazole, ketoconazole, and lincomycin are present in the composition for about 0.5%, 0.2%, and 1.8% (w/v), respectively.

In some embodiments, the metronidazole, ketoconazole, and lincomycin are present in a synergistic ratio. For example, in some embodiments, the weight ratio of metronidazole and ketoconazole in the composition is about 1:5 to about 5:1, including for example about any of 1:2, 1:1, 2:1, 3:2, 5:2, or 5:1. In some embodiments, the weight ratio of lincomycin and ketoconazole in the composition is about 1:10 to a bout 10: 1, including for example about any of 1:5, 1:1, 2:1, 3:1, 4:1, 5:1, 6:1, 7:1, 8:1, 9:1, or 10:1. In some embodiments, the weight ratio of metronidazole, ketoconazole, and lincomycin in the composition is about 2.5:1:9. In some embodiments, the ratio of metronidazole, ketoconazole, and lincomycin in the composition is about 2:5:1:4.5.

The composition comprising metronidazole, ketoconazole, and lincomycin can be in any suitable forms. For example, in some embodiments, the composition is in the form of a suspension. In some embodiments, the composition is in the form of an aqueous gel, such as an aqueous alcohol gel. In some embodiments, the composition is in the form of a cream. In some embodiments, the composition is in the form of an ointment. In some embodiments, the composition is in the form of an emulsion. In some embodiments, the composition is in the form of a liposomal composition.

The composition can further comprise a pharmaceutically acceptable carrier. Any dermatologically acceptable carrier can be used in the compositions of the invention. As used herein, “dermatologically acceptable carrier” refers to vehicles, diluents, carries, which can include adjuvants, additives, or excipients, known for use in dermatological compositions. The compositions of the invention include, but are not limited to, creams, ointments, solutions, lacquers, sticks, pledgets, wipes, cleansers and/or gels.

In one embodiment, the composition is a solution that can be used as a cleanser. In another embodiment, the composition can be used as a lotion.

In yet another embodiment, the topical composition is a semi-solid at room temperature but is easily absorbed into the stratum comeum. The semi-solid composition can be a cream. Such a composition can include petroleum-based liquids and solid fractions as skin protectants. The solid skin protectant can be semi-solid. The solid skin protectant can be present in about 5.5% to about 20% in the composition and includes petrolatum or a synthetic or semi-synthetic hydrocarbon of the same nature as petrolatum. Mixtures of such ingredients can also be used. Liquid skin protectants can be petrolatum and contained in the composition in about 10% to about 20% and include any synthetic or semi-synthetic oleaginous liquid fraction. The liquid skin protectant can be mineral oil, which is a liquid mixture of hydrocarbons obtained from petroleum.

Other anti-inflammatory agents can also be incorporated into the formulation at therapeutic levels like for example corticosteroids and nonsteroidal anti-inflammatory drugs (NSAIDs). Examples of corticosteroids are betamethasone, clobetasol, diflorasone, halobetasol, amcinonide, desoximetasone, diflorasone, fluocinolone, halcinonide, clocortolone, desoximetasone, flurandrenolide, fluticasone, hydrocortisone, mometasone, triamcinolone, aclometasone, desonide, dexamethasone, fluocinolone and their pharmaceutically acceptable salts. Examples of nonsteroidal anti-inflammatory drugs (NSAIDs) are flurbiprofen, diclofenac, metronidazole, ketorolac and their pharmaceutically acceptable salts.

### Methods of Treating Acne

The compositions described herein are particularly suitable for treating or preventing infectious skin disease (such as acne) in an individual. Infectious skin diseases include, but are not limited to, psoriasis, seborrhea, rosacea, ingrown hairs, pseudofolliculitis barbae, hyperpigmented skin, and cutaneous infection.

One preferred route of administration is topical administration. The term “topical administration” is used in its conventional sense to mean application of an active agent to the skin or mucosa to achieve a local effect.

In some embodiments, the method further comprises ionized vaporation of the infected area. In some embodiments, the composition is applied in conjunction with ultrasound.

“Individual” used herein includes human. In some embodiments, the individual is less than about any of 20, 30, 40, or 50 years old. In some embodiments, the individual is prone to develop acne. In some embodiments, the individual has acne.

“Treating” or “to treat” an infectious skin disease using methods of the invention is defined as administering the composition of the present invention, in order to palliate, ameliorate, stabilize, reverse, slow, delay, reduce, or eliminate either the infectious skin disease or visible lesions (or other symptoms) of the infectious skin disease. “Treating” or “to treat” an infectious skin diseases also encompasses reducing frequency of occurrence and/or preventing, delaying, or reducing frequency of recurrence of the infectious skin disease or visible lesions (or other symptoms) of the infectious skin disease. A “therapeutically effective amount” or “an amount sufficient to have a therapeutic effect” is an amount sufficient to treat an infectious skin disease, as defined above. An effective amount can be administered in one or more administrations.

To “prevent” an infectious skin disease using methods of the invention is defined as administering a composition of the present invention in order to suppress the infectious skin disease or visible lesions (or other symptoms) of the infectious skin disease. Prevention can be partial or total.

In some embodiments, the composition of the present invention is used to treat visible lesions of an infectious skin disease. Visible lesions of an infectious skin disease (such as acne) include closed comedones, open comedones, red or pustular-looking inflamed papules, pustules, nodules and cysts of acne or cutaneous infection; visible ingrown hairs of pseudofolliculitis barbae; visible scales of seborrhea, ichthyosis and psoriasis; and the like. Visible lesions can be due to obstruction of follicular ducts, thickened sebum, bacterial infection, or a combination thereof. Accordingly, the compositions can be used to prevent obstruction of follicular ducts, to reopen a duct if it has become blocked, to combat thickened sebum, to combat bacterial infection, or a combination thereof. Treatment of visible lesions can be evaluated based on the effectiveness of the treatment in reducing the number and severity of visible lesions. Any reduction in number or severity of visible lesions as a result of administration a composition would be considered treatment of visible lesions. In some embodiments, the nodules or cysts of acne (or other visible lesions) are physically removed prior to application of the composition.

In one embodiment, the composition of the invention is used to treat pre-emergent lesions. As used herein, “pre-emergent lesions” refers to non-visible lesions present within the skin prior to eruption of visible lesions on the surface of the skin. Like visible lesions, pre-emergent lesions can be due to obstruction of follicular ducts, thickened sebum, bacterial infection, or a combination thereof. Accordingly, the compositions can be used to treat pre-emergent lesions by preventing obstruction of follicular ducts, reopening a duct if it has become blocked, combating thickened sebum, combating bacterial infection, or a combination thereof. While pre-emergent lesions are insufficiently visible to be graded in conventional clinical studies, their presence within the skin can be discerned by the tactile sense of feel and/or by pain and tension within the skin. Any reduction in number of locations within the skin in which pre-emergent lesions exist as a result of administration of a composition would be considered treatment of pre-emergent lesions. Similarly, any reduction in the severity of the symptoms of a pre-emergent lesion as a result of administration of a composition would be considered treatment of the pre-emergent lesion.

In another embodiment, the compositions of the invention can be used to treat acne. As used herein, “acne” means a disorder of the skin caused by inflammation of skin glands or hair follicles. The compositions of the invention can be used to treat acne at early pre-emergent stages or later stages where lesions from acne are visible. Early pre-emergent stages of acne usually begin with an excessive secretion of sebum or dermal oil from the sebaccous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone. In the normal sequence of developing acne, hyperkeratinazation of the follicular opening is stimulated, thus completing blocking of the duct. The usual results are papules, pustules, or cysts, often contaminated with bacteria, which cause secondary infections. Acne is characterized particularly by the presence of comedones, inflammatory papules, or cysts. The appearance of acne may range from slight skin irritation to pitting and even the development of disfiguring scars. Accordingly, the compositions of the invention can be used to treat skin irritation, pitting, development of scars, comedones, inflammatory papules, cysts, hyperkeratinazation, and thickening and hardening of sebum associated with acne and rosacea.

The desired amount of the ingredients in the composition can vary from composition to composition depending on the particular disorder being treated, the severity of the disorder, the duration of the treatment, and other specific components being used.

In some embodiments, the composition described herein is applied topically to the skin at least once a day, including for example at least about any of two times a day, three times a day, four times a day, or more frequently. In some embodiments, for example when the composition is administered in conjunction with ultrasound, the composition is administered less frequently, including for example about any of once every two days, once every three days, once every four days, once every five days, once every six days, once a week, or once every two weeks.

### Kits and Unit Dosages

Also provided herein are kits for methods described herein.

In some embodiments, there is provided a kit comprising metronidazole, ketoconazole, and lincomycin. The three components can be stored in the same or different packages. For example, in some embodiments, the kit comprises metronidazole, ketoconazole, and lincomycin in separate packages, and the three ingredients are mixed together to make the composition of the present invention prior to application of the composition. Once mixed, the composition can be stored for up to four weeks prior to the application of the composition.

Suitable containers include, for example, bottles, vials, and test tubes. The containers may be formed from a variety of materials such as glass or plastic.

In some embodiments, the kits further comprise instructions on making of the composition and/or use of the composition for treating infectious diseases.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

The following Examples are provided to illustrate, but not limit, the invention.

### EXAMPLE 1

This example provides one exemplary formulation of compositions of the present invention and use thereof for treating acne.

0.2 grams of ketoconazole was dissolved in 100 ml metronidazole (0.5%) injection solution. 1.8 grams lincomycin injection solution was then added to the mixture to make a suspension. The suspension can be stored for up to about 5 days.

For treatment of acne, apply the suspension to affected areas of an individual at least two times a day. Acne disappears within about 1 day.

### EXAMPLE 2

This example provides exemplary methods of applying compositions of the present invention.

1081 patients who were diagnosed with acne were divided into two groups. The first group had 729 patients, 278 males, and 451 females. The ages of the patients ranged from 12 years old to 48 years old. The average age was about 25 years old. The patients had acne for about 1 month to about 8 years, with an average of about 2.5 years. [classifications] All patients had acne infections on the face. 98 patients also have acne infection on the chest; 37 on the back. 10 patients had acne on face, chest, and back. None of the patients had systemic disease. None of them were pregnant or breast feeding.

The second group had 352 patients.

Patients in the first group were treated as follows. The facial skin was first washed with soap, and covered with the composition described in Example 1. Ionized vapor spray was applied to the face for 10 minutes, nodules were then removed by using sterilized needles. The composition was again applied to the infected area by using ultrasound. GP-728 ultrasounic cosmetic machine was used for this method, with a frequency of 10 KHz and power of 0-10 W. The ultrasound intensity was 0.5-1 W/cm2, under a continuous mode. After 8-10 minutes of treatment, the face was covered with a face membrane for 20 minutes and washed thoroughly. The method was repeated twice a week for four weeks.

The second group was treated by directly applying the composition described in Example 1 to the affected areas twice daily for four weeks.

The criteria for treatment is based on the following standard. Complete efficacy: skin eruption disappeared; High efficacy: 60% or more decrease in skin eruption without new skin eruptions; efficacy: 30% or more decrease in skin eruption, with occasional new skin eruptions; no efficacy: no apparent change in skin eruption with new skin eruptions. The results of the example is provided in Table 1. Treatment efficiency is based on the total of patients with complete or high efficacy.

TABLE 1

| | Patient | Complete | High | | No | Treatment |
|---|---|---|---|---|---|---|
| Group | No. | efficacy | efficacy | Efficacy | efficacy | efficiency |
| 1 | 729 | 386 | 258 | 69 | 16 | 88.34% |
| | | (52.95) | (35.39%) | (9.47%) | (2.19%) | |
| 2 | 352 | 68 | 185 | 67 | 32 | 71.88% |
| | | (19.32%) | (52.57%) | (19.03%) | (9.09%) | |

What is claimed is:

1. A composition for treatment of an infectious skin disease in an individual by topical administration, comprising metronidazole, ketoconazole, and lincomycin.

2. The composition according to claim 1, wherein the composition is a liquid suspension.

3. The composition according to claim 2, wherein metronidazole in the liquid suspension is about 0.1%-2% (w/v).

4. The composition according to claim 2, wherein the ketoconazole in the liquid suspension is about 0.05% to about 0.5% (w/v).

5. The composition according to claim 2, wherein the lincomycin in the liquid composition is about 0.5% to about 5% (w/v)

6. A method of treating acne in an individual, comprising topically administering to the affected area of the individual an effective amount of a composition of claim 1.

7. The method of claim 6, further comprising applying ionized vapor to the affected area prior to the administration of the composition.

8. The method of claim 6, further comprising the step of removing the nodule prior to administration of the composition.

9. The method of claim 6, wherein the composition is administered with the aid of ultrasound.

10. The method of claim 6, wherein the individual is less than about 30 years old.
